(19) United States
(12) Patent Application Publication
(10) Pub. No.: US 2005/0096684 A1
(43) Pub. Date: May 5, 2005

(54) Programmable surgical instrument system

(75) Inventors: Wade P. Farrow, Bryan, TX (US); E. Paul Strauss, Grapevine, TX (US); Robert Ellins, Euless, TX (US); John Murphy, Haltom City, TX (US); Samuel Owusu-Akyaw, Southlake, TX (US)

Correspondence Address: HAYNES AND BOONE, LLP, 901 MAIN STREET, SUITE 3100, DALLAS, TX, 75202, US

(73) Assignee: Medtronic, Inc., Minneapolis, MN (US)

(21) Appl. No.: 10/699,474

(22) Filed: Oct. 31, 2003

(51) Int. Cl. A61B 1/06; A61B 17/32

(52) U.S. Cl. 606/170; 600/170

(57) ABSTRACT

A surgical tool system comprises a first hand instrument including a first signature discrete circuit element and a control console connected to the first hand instrument. The control console includes a memory device and instructions for identifying and controlling a plurality of hand instruments, including the first hand instrument. The first signature discrete circuit element is detected by the control console for identifying the first hand instrument.

### BACKGROUND

To improve the speed and accuracy of surgical procedures, surgeons and other medical professionals often use powered surgical instruments for dissecting bone and tissue. While various powered instruments are known in the art, existing systems have certain disadvantages. For instance, hand instruments may comprise delicate integrated circuits which exhibit poor reliability after the instruments are exposed to repeated sterilization procedures. Current systems may also have little or no capacity to upgrade instrument control software as new instruments are introduced or to adapt the instrument control software to accommodate particular user applications. Thus, current systems may require expensive and disruptive replacement of hand instrument and console components or even replacement of the entire system. Accordingly, a need exists in the pertinent art for more robust hand instruments and for more flexible instrument control components.

### SUMMARY

A surgical tool system comprises a first hand instrument including a first discrete signature identification circuit element and a control console connected to the first hand instrument. The control console includes a memory device and instructions for identifying and controlling a plurality of hand instruments, including the first hand instrument. The first discrete signature identification circuit element is detected by the control console for identifying the first hand instrument.

In another embodiment, the first hand instrument is without integrated non-volatile memory.

In another embodiment, the control console further comprises an interface for transferring the instructions from a download source.

Still another embodiment comprises a method of operating a surgical tool system having a control console connected to a first hand instrument. The method comprises identifying the first hand instrument connected to the control console and responsive to the identification of the first hand instrument, selecting first instructions for adapting the control console for controlling the first hand instrument. The method further comprises controlling the first hand instrument with the selected first instructions. Identifying the first hand instrument comprises recognizing a first discrete signature identification circuit element included in the first hand instrument.

### BRIEF DESCRIPTION OF THE DRAWINGS

FIG. 1 illustrates a surgical instrument system according to one embodiment of the present invention.

FIG. 2 illustrates a hand instrument according to one embodiment of the present invention.

FIG. 3 is a process for operating a hand instrument according to one embodiment of the present invention.

FIG. 4 is a schematic diagram of a feedback network according to one embodiment of the present invention.

FIG. 5 illustrates a control software reference table according to one embodiment of the present invention.

FIG. 6 is a graph of a first set of operational parameters for the surgical instrument system of FIG. 1.

FIG. 7 is a graph of a second set of operational parameters for the surgical instrument system of FIG. 1.

FIG. 8 is a process for upgrading control software according to one embodiment of the present invention.

### DETAILED DESCRIPTION

The present invention relates generally to a surgical instrumentation and more particularly to an upgradeable and programmable surgical instrument system. For the purposes of promoting an understanding of the principles of the invention, reference will now be made to the embodiments or examples illustrated in the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

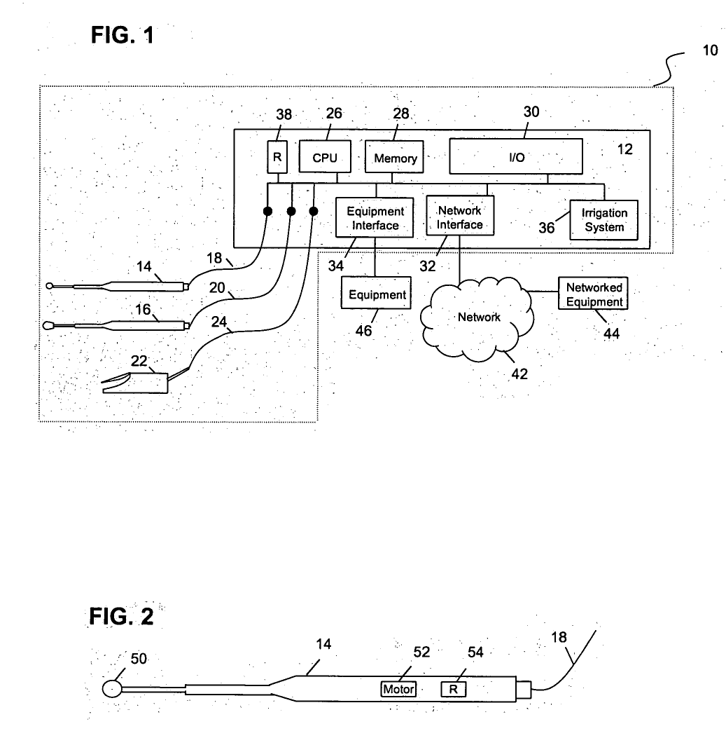

Referring first to FIG. 1, the numeral 10 refers to an exemplary surgical instrument system which may include a control console 12 for electrically powering and controlling hand instruments 14, 16 which may be connected to the control console 12 by cables 18, 20 respectively. Although two hand instruments are described, it is understood that the system 10 may include any number of hand instruments with corresponding cables attached to the console. Other control instruments such as a foot control 22, connected to console 12 by a cable 24, may also be included. The control console 12 may include a central processing unit (“CPU”) 26, a memory unit 28, an input/output (“I/O”) device 30, a network interface 32, an equipment interface 34, an irrigation system 36, and at least one recognition circuit element 38. The components 14-38 may be interconnected by a bus system 40.

It is understood that the surgical instrument system 10 may be differently configured and that each of the listed components may actually represent several different components. For example, the CPU 26 may actually represent a multi-processor or a distributed processing system, and in one embodiment the CPU 26 may be a digital signal processor. The memory unit 28 may include different levels of cache memory, main memory, hard disks, remote storage locations, and removable storage devices which may further include CD-ROMs and floppy disks. The I/O device 30 may include monitors, touch screens, keyboards, and other integrated or non-integrated devices which inform a user of and/or permit a user to alter current system parameters and hand instrument operation characteristics. The recognition circuit element 38 may be a resistor.

The surgical instrument system 10 may be connected to a network 42. The network 42 may be, for example, a subnet of a local area network, an organization wide intranet, and/or the Internet. Through the network 42, the surgical system 10 may be connected to networked equipment 44 which may be, for example, a remote diagnostic computer, a server for hosting a website, or a remote software upgrade system. The surgical instrument system 10 may be further connected to equipment 46 through the equipment interface 38. The equipment 46 may be diagnostic equipment or a personal computer. The equipment interface 34 may comprise a serial port, such as a RS 232 port, for connecting to the equipment 46.

The operation of the surgical instrument system 10 may be controlled by control software (not shown) which may reside on the CPU 26 of control console 12. The control software for the control console 12 may, for example, include instructions for signaling and receiving signals from the I/O device 30; providing drive signals to and receiving feedback signals from the hand instruments 14, 16; and receiving feedback signals from the foot control 22.

The surgical instrument system 10 may accept any of a variety of console driven powered surgical instruments, including instruments designed for small bone, large bone, arthroscopic or laproscopic procedures. As shown in FIG. 2, the hand instrument 14 may include a dissection instrument 50 driven by a motor 52. The hand instrument 14 may further include a signature discrete circuit element 54, such as a resistor or zener diode, for establishing the existence and the type of hand instrument connected to the console 12. It should be understood that the signature discrete circuit element 54 may comprise a plurality of discrete circuit elements. For example, the signature discrete circuit element 54 may be a single resistor or a plurality of resistors, and each hand instrument type may be uniquely identified by a different signature resistor. In some embodiments, the signature resistor 54 may be attached to a printed circuit board (not shown) within the hand instrument 14. The hand instrument 14 may be designed for small bone applications using instruments such as a high speed drill, a high torque instrument, a sagittal saw, an oscillating saw, a reciprocating saw, or a micro drill. Hand instrument 16 may be similarly configured and therefore will not be described in detail.

The hand instrument 14 may provide feedback signals to the software of the control console 12 which, in turn, are used to determine the control signals that are applied to the hand instrument 14. These control signals may include variable voltage signals, such as one to identify the hand instrument type as described below and feedback signals used to determine the speed of the rotation. Digital control signals may also be available. The digital signals may determine the operation mode of the hand instrument such as forward, reverse, or oscillate. The digital signals may be interpreted differently for each hand instrument based upon the identified signature resistor 54 or based upon user inputs.

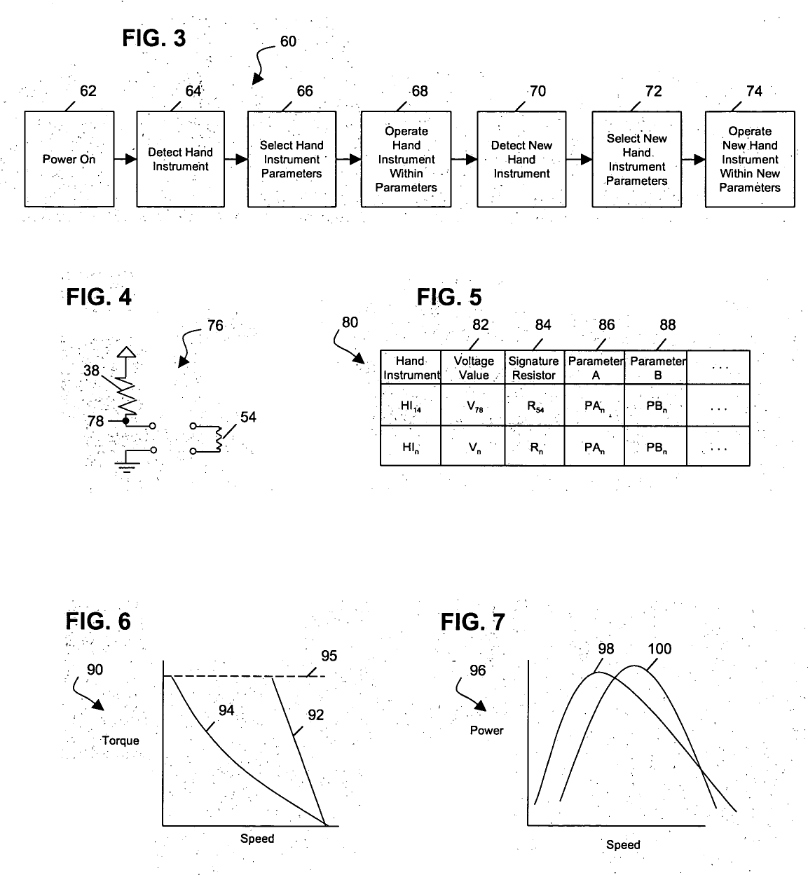

Referring now to FIG. 3, a hand instrument set-up and operation process 60 may be implemented using the instructions provided by the software of the control console 12 to detect the connection of a particular type of hand instrument and to prepare the particular type of hand instrument for operation. At step 62, the power to the control console 12 may be turned on and the CPU 26 may be initialized.

At step 64, the control console 12 may check for the connection of one or more hand instruments by passing a current through a feedback network (FIG. 4) and measuring a voltage value. FIG. 4 illustrates an exemplary feedback network 76 comprising the recognition resistor 38 of the control console 12 serially connected with the signature resistor 54 of the hand instrument 14. FIG. 4 illustrates that signature resistor 54 may be disconnected from the feedback network 76, and in other embodiments, alternative types of hand instruments, having unique signature resistors may be connected to the control console 12. With the feedback network 76 configured as in FIG. 4, a voltage 78 measured between signature resistor 54 and recognition resistor 38 may be compared to a stored reference table (FIG. 5) that relates voltage values to hand instrument types. If is understood that the feedback network may be differently and may have different or additional components which may be located in the control console 12 or the hand instrument 14. Also, the measured voltage maybe determined at a different point in the reference feedback network.

FIG. 5 illustrates a stored reference table 80 which may be incorporated into or accessible by the control software of the control console 12. The reference table 80 may correlate a hand instrument type to a plurality of fields such as a measured voltage value field 82, a signature resistor value field 84, a first operational parameter 86 and a second operational parameter 88. Using the measured voltage 78, the control console 12 may consult the reference table 80 to identify the connected hand instrument as hand instrument 14. Each hand instrument type may have a different signature resistor and thus, the measured voltage may differ with different hand instrument types. The measured voltage may also indicate that no hand instrument is connected or that a short has occurred in the circuit.

By using the feedback network, hand instruments may be manufactured without integrated circuits or other volatile or non-volatile memory storage devices for identifying the hand instruments. This absence of delicate integrated circuitry and stored data can help to maintain the reliability of the hand instruments and avoid the need for replacing hand instruments and/or integrated circuits even after repeated exposure to the harsh environment of a steam autoclave and/or other sterilization processes.

Referring again to FIG. 3, at step 66 a set of operating parameters corresponding to the identified signature resistor 54 may be selected from parameter fields 86, 88 of the reference table 80 to operate the identified hand instrument 14. The drive parameter fields 86, 88 may comprise motor drive control algorithms incorporated into or accessible by the control software of the console 12 and tailored to the specific function of each hand instrument type. The control algorithms may, for example, correspond to torque, speed, and/or power curves. FIG. 6 illustrates a set of speed/torque curves 90. The individual curves 92, 94 may be modified and optimized for specific hand instrument types. Each speed/torque curve may correspond to a specific hand instrument identified through feedback network 76 and the reference table 80, such as curve 92 corresponding to hand instrument 14. In this example, the curve 92 may govern the speed/torque performance characteristics of the hand instrument 14. Curve 94 may correspond to an additional or an alternative hand instrument type. A cut-off point 95 may establish a limit within which an infinite number of curves may be defined. The cut-off point 95 may be a point which limits current draw by the system to prevent electric motor overload and overheating.

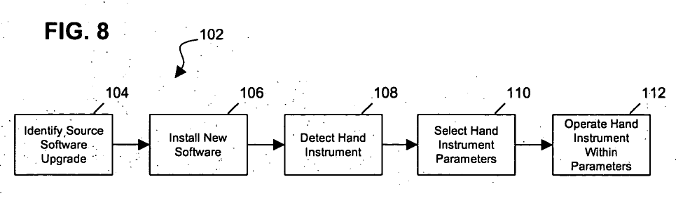

FIG. 7 illustrates a set of speed-power curves 96. The individual curves 98, 100 may be modified and optimized for specific hand instrument types. These modifications may be provided to the, control console using the software upgrade process described below in reference to FIG. 8. Each speed-power curve may correspond to a specific hand instrument identified through the feedback network 76 and the reference table 80, such as curve 98 corresponding to hand instrument 14. In this example, the curve 74 may govern the speed-power performance characteristics of the hand instrument 14. Curve 100 may correspond to an additional or an alternative hand instrument. The ability to modify and optimize the operating curves 90, 96 may be limited to certain users of the control console 12 such as maintenance users.

The parameters described in FIGS. 6 and 7 are merely exemplary and it is understood that control of the hand instruments connected to the control console may be governed by alternative or additional algorithms, performance curves, or other performance data, including motor speed-current data. Furthermore, the drive parameters may be specifically tailored to a particular customer application. Some embodiments may, after the hand instrument type is identified, allow a user to select from a plurality of parameters associated with the identified hand instrument, permitting the user to tailor the parameters to a particular patient or surgical procedure. In one embodiment, the control console may automatically switch between alternative curves in response to user inputs to the system 10. In another embodiment, the control console 12 may operate the hand instrument 14 in such a way as to mimic the torque-speed curve of a pneumatically powered hand instrument. In this way, the hand instrument 14 may mimic the torque-speed curve of any variety of hand instruments provided that the torque-speed curve does not exceed the maximal output of the control console and hand instrument combination. In one example where the hand instrument 14 is a high speed drilling instrument, the torque of the instrument may be at least partially programmable by the user. The ability to program the drilling instrument may be flexible within the parameters associated with the signature resistor of the drilling instrument. Using an I/O device 30 on the console 12, the user may, for example, select an upper torque limit. The selected limit may be displayed in terms of a percentage of the maximum hand instrument torque.

At step 68, the hand instrument 14 may be operated within the parameters 92, 98 which the control console 12 recognizes to be associated with the signature resistor 14. At step 70, the control console 12 may continuously monitor or periodically poll the feedback network 76 to determine whether a new signature resistor belonging to a new hand instrument type has become connected to the control console 12. The presence of a new hand instrument may be detected, as described above, by passing a current through the feedback network 76 and measuring the new resulting voltage. The new measured voltage may be compared to the voltage values 82 stored in reference table 80. Based upon the reference table 80, the control console 12 may identify the new hand instrument. At step 72, the control console 12 may select the set of stored parameters corresponding to the newly identified signature resistor, for driving the motor of the new hand instrument. At step 74, the new hand instrument is operated within the corresponding parameters.

As powered surgical instrumentation technology advances and new instruments are developed, the product life cycle of the control console 12 may be extended by upgrading the software of the control console 12 to recognize new hand instrument types or to permit new operating parameters for existing hand instrument types. Upgrading the software may include replacing, modifying, or supplementing the existing software of the control console 12. The ability to upgrade the control console 12 allows the user of the system instrument system 10 to use the most modern instrumentation without purchasing a new console 12 and also allows the user to receive performance algorithms tailored specifically to the user's needs. The upgradeable control console 12 may also allow the user to receive the software upgrade from a variety of different sources through a variety of different mechanisms.

Referring now to FIG. 8, a process 102 for upgrading the software of the control console 12 may begin at step 104 with the identification of the source for the software upgrade. In one embodiment, the software upgrade may be received by the control console 12 from the equipment 46 connected to control console 12. The software may be downloaded from the equipment source 46 to the CPU 26 of the control console 12. For example, the equipment 46 may be a personal computer which may be directly connected to the control console through the equipment interface 34. In an alternative embodiment, the software for the control console 12 upgrade may be provided by adding or replacing memory unit 28. For example, a floppy disk or a CD-ROM may be provided for directly uploading new algorithms and other control parameters to the control console 12. In still another alternative, the software upgrade may be transmitted to the control console 12 through the network interface 32. For example, the control console 12 may connected to the Internet 42 via the network interface 32. Through the Internet 42, the console 12 may access a web site hosted by a remote server 44 to download the software upgrade. The control console 12 may periodically access the web site hosted by the server 44 to obtain software upgrades. In certain other embodiments, the remote server 44 may remain in communication or make contact with the control console 12 through the Internet 42 to provide software upgrades as they become available to the remote server 44.

After identifying the source of the software upgrade, the software upgrade may be installed at step 106. In this embodiment, the software upgrade may, for example, allow the control console to identify additional signature resistors corresponding to new hand instrument types. Additionally, the software upgrade may introduce operational parameters associated with the new hand instrument types. Proceeding to step 86, a new hand instrument may be introduced to the control console which was not recognized by the control console prior to the software upgrade installation. After the upgrading of the software to include information about the new hand-instrument type, the control console 12 may poll the feedback network 76 to determine the identity of the new hand instrument. Then at step 88, the parameters corresponding to the new hand instrument may be selected. At step 90, the new hand instrument may be operated within the selected parameters.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. In the claims, means-plus-function clauses are intended to cover the structures described herein as performing the recited function and not only structural equivalents, but also equivalent structures.

1. A surgical tool system, the system comprising:
a first hand instrument including a first signature discrete circuit element;
a control console connected to the first hand instrument, the control console including a memory device and instructions for identifying and controlling a plurality of hand instruments, including the first hand instrument,
wherein the first signature discrete circuit element is detected by the control console for identifying the first hand instrument.

2. The surgical tool system of claim 1 wherein the first signature discrete circuit element is a first signature resistor.

3. The surgical tool system of claim 1 wherein the control console further comprises an interface for transferring the instructions from a download source.

4. The surgical tool system of claim 3 wherein the download source is diagnostic equipment.

5. The surgical tool system of claim 3 wherein the download source is a personal computer.

6. The surgical tool system of claim 3 wherein the download source is a removable storage device.

7. The surgical tool system of claim 3 wherein the instructions are transferred over a network.

8. The surgical tool system of claim 1 wherein the instructions for controlling the plurality of hand instruments include instructions for controlling the torque-speed curve of the first hand instrument.

9. The surgical tool system of claim 1 wherein the instructions for controlling the plurality of hand instruments include instructions for controlling the torque of the first hand instrument.

10. The surgical tool system of claim 1 wherein the instructions for controlling the plurality of hand instruments include instructions for controlling the power to the first hand instrument.

11. The surgical tool system of claim 1 wherein the control console controlling of the hand instrument mimics a torque-speed curve of a pneumatically powered hand instrument.

12. The surgical tool system of claim 1 wherein the control console mimics the torque-speed curve of any of a plurality of hand instruments without exceeding a maximal output of the control console and the hand instrument.

13. The surgical tool system of claim 1 wherein the first hand instrument is without integrated non-volatile memory.

14. The surgical tool system of claim 1 wherein the first hand instrument is without integrated memory.

15. The surgical tool system of claim 1 further comprising a second hand instrument including a second signature discrete circuit element, wherein the instructions for identifying and controlling a plurality of hand instruments include instructions for identifying and controlling the second hand instrument and wherein the second signature discrete circuit element is detected by the control console for identifying the second hand instrument.

16. A method of operating a surgical tool system having a control console connected to a first hand instrument, the method comprising:
identifying the first hand instrument connected to the control console;
responsive to the identification of the first hand instrument, selecting first instructions for adapting the control console for controlling the first hand instrument;
controlling the first hand instrument with the selected first instructions;
wherein identifying the first hand instrument comprises recognizing a first signature circuit element included in the first hand instrument.

17. The method of claim 16 wherein the first signature circuit element is a first signature resistor.

18. The method of claim 16 wherein the first signature circuit element is a first signature zener diode.

19. The method of claim 16 wherein identifying and controlling the first hand instrument is accomplished by the control console without reading stored data from the first hand instrument.

20. The method of claim 16 further comprising:
connecting the control console to a download source;
downloading upgrade instructions for modifying the first instructions;
controlling the first hand instrument with the modified first instructions.

21. The method of claim 16 wherein recognizing the first signature circuit element includes passing a current through a feedback network, wherein the feedback network comprises the first signature circuit element of the first hand instrument and a recognition circuit element of the control console.

22. The method of claim 21 further comprising:
polling the feedback network to detect a disconnection of the first signature circuit element and a connection of a second signature circuit element corresponding to a second hand instrument;
responsive to the detection of the connection of the second signature circuit element, selecting second instructions for adapting the control console for controlling the second hand instrument; and
controlling the second hand instrument with the selected second instructions.

23. The method of claim 16 further comprising:
connecting the control console to a download source;
downloading third instructions from the download source for identifying and controlling a third hand instrument; disconnecting the first hand instrument from the control console;
connecting the third hand instrument to the control console;
identifying the third hand instrument connected to the control console;
responsive to the identification of the third hand instrument, selecting third instructions for adapting the control console for controlling the third hand instrument;
controlling the third hand instrument with the selected third instructions.

24. A surgical tool system, the system comprising:
a hand instrument without integrated non-volatile memory, the hand instrument comprising a motor for driving a dissection instrument;
a control console connected to the hand instrument, the control console including software for identifying the hand instrument and for providing a first set of hand instrument control parameters; and
a second set of hand instrument control parameters for replacing the first set of control parameters during an upgrade of the software.

25. The surgical tool system of claim 24, the system further comprising:
a feedback network for identifying the hand instrument, the feedback network comprising a first resistor in the hand instrument and a second resistor in the control console.

26. A control console, the console comprising:
a connector for connecting an instrument;
identification software for identifying a signature resistor in the instrument;
control software for maintaining a plurality of sets of operational parameters for controlling a plurality of instruments;
selection software for matching the identified signature resistor to one of the plurality of sets of operational parameters; and
operation software for operating the instrument within the selected set of operational parameters.

27. The control console of claim 26 wherein
the selected set of operational parameters includes data corresponding to a performance curve and wherein the operational software operates the instrument along the performance curve.
